(19) United States
(12) Patent Application Publication
(10) Pub. No.: US 2011/0203586 A1
(43) Pub. Date: Aug. 25, 2011

(54) Powder Inhalers

(75) Inventors: Marc Egen, Ingelheim am Rhein (DE); Michael Krueger, Ingelheim am Rhein (DE); Joerg Schiewe, Mainz (DE)

(73) Assignee: BOEHRINGER INGELHEIM INTERNATIONAL GMBH, Ingelheim am Rhein (DE)

(21) Appl. No.: 12/918,094

(22) PCT Filed: Feb. 20, 2008
(86) PCT No.: PCT/EP08/52085

§ 371 Date: Feb. 14, 2011

(30) Foreign Application Priority Data
Feb. 21, 2008 (JP) 2008-040229

(51) Int. Cl. A61M 15/00

(52) U.S. Cl. 128/203.15

(57) ABSTRACT

The invention relates to a powder inhaler for administering moisture-sensitive pharmaceutical formulations.

The invention relates to a powder inhaler for administering moisture-sensitive pharmaceutical formulations.

### PRIOR ART

Medical aerosol therapy directed to pulmonary inhalation by means of a nebuliser, metered-dose aerosol or dry powder inhaler plays an important role in the treatment of a number of diseases and particularly diseases of the respiratory tract.

In the field of powder inhalers, single-dose and multi-dose devices are known. These may take the form of single-use or reusable devices. In single-dose powder inhalers, the dosing may take the form of capsules which contain a powder formulation. If a capsule is used as the container, this is opened in the powder inhalers by piercing, crushing or cutting before the inhalation manoeuvre, so that the powder can be moved out of the capsule by the patient's breath and produce an airborne aerosol which the patient breathes in. A distinction is also drawn between multi-dose powder inhalers which contain the formulation in the form of a powder supply, from which the respective single dose is taken using a built-in dosing unit, and powder inhalers with pre-dosed packaged single doses.

Examples of inhalers designed according to these two principles are known in the art.

One example of a single-dose powder inhaler is the HandiHaler®, as disclosed e.g. In EP 1342483.

DE 3348370 and DE 3336486 disclose inhalers which contain a disc-shaped blister pack comprising a number of wells arranged in a circle. The individual wells each contain a dose of a powdered medicament intended for inhalation. The wells are closed off on one side by a sealing film, for example. To deliver the powdered medicament, the well is opened. An air channel connects the opened well to the mouthpiece of the inhaler. The inhaler of DE 3336486 will be described in more detail by way of example. It comprises a housing containing a chamber (supply chamber) which has an air inlet and in which there is a disc-shaped round blister with filled pouches of medicament. The blister is loosely attached to a round rotatable disc. Holes are formed around the disc which come into contact with the medicament pouches in the axial direction, i.e. The pouches and holes are arranged above and below one another. The chamber has an air outlet. The inhaler also has a plunger which is arranged so that it can pierce a medicament pouch to open it, allowing the medicament to be released into the chamber and breather in through a mouthpiece. The drawings in the patent application or the US patent specification may be consulted, to which reference is hereby specifically made.

DE 4106379 describes an inhaler into which a blister or the like for a powdered medicament may be introduced. The blister consists of two strips of material that can be detached from one another, defining at least one container in which the medicament is found. The device is provided with means for opening the container by pulling the two strips of material apart at an opening station. The user can inhale the powdered medicament from the opened container through an outlet part, e.g. a mouthpiece, which is connected to the opened container. One of the strips of material may also be a carrier strip comprising a number of pouches and the other strip of material may be a cover strip. Each pouch and the adjacent part of the cover strip then forms a container. At the opening station, a drive device may be provided which pulls the carrier strip and the cover strip apart. This drive device consists for example of two drive wheels (e.g. cogs) which hold the cover strip in driving engagement between them. In this case, too, each individual blister defines a kind of storage chamber in the inhaler, which is connected to the mouthpiece via an air channel.

In any case, the manner is which the powder formulation is packaged in the device is critical to the quality of the product and hence its suitability for use by inhalation.

With regard to the packaging of medicament powders, a distinction is made between the primary packaging and the secondary packaging.

The primary packaging is characterised in that it is in direct contact with the inhalable formulation.

The primary packaging may optionally be surrounded by a second, outer protection, the secondary packaging.

The primary packaging may be, for example, a capsule, a solid or flexible blister with wells or a disc comprising wells.

The secondary packaging may be a blister, a pouch, a bag or other container. The secondary packaging generally totally encloses the primary packaging. Secondary packaging is used particularly when the primary packaging does not provide adequate protection from moisture.

The secondary packaging, such as a single- or multi-dose powder inhaler, for example, is made from standard commercial plastics.

The primary packaging and optionally the secondary packaging have the task of protecting the active substance and also the entire inhalable formulation from chemical or physical change. The physical changes in question may be, in particular, changes to the cohesion of the active substance particles, changes to the adhesion of active substance particles to excipients and container walls, or a water-induced chemical decomposition, all of which might affect the delivery of the intended dose of fine particles. By the term “dose of fine particles” is meant the dose of pharmaceutical formulation which reaches the patient's lungs. It is affected by the interactions of the micronised active substance particles with one another and also the interactions with the excipients or with the container walls. It has been found that changes in the moisture level inside the packaging in particular may increase these interactions to such an extent that the fine particle dose is significantly reduced. Such changes include the penetration of water into the packaging as well as the removal of water from the interior of the packaging.

A primary objective of the packaging is therefore to keep the chemical composition of the atmosphere inside the packaging constant so as to prevent physical or chemical changes to the active substance formulation, or to keep the inhalable formulation stable. In this context a distinction is made between the “in-use stability” and the long-term stability. The former is a stability directed to a short time which the inhalable formulation must have per se, even if it is not adequately protected by the packaging. The long-term stability is defined as the stability that has to be guaranteed as long as the inhalable formulation is inside the unopened packaging.

The choice of a suitable material for the secondary packaging is determined by two factors:
On the one hand the material must be able to fulfil the protective function described.
On the other hand the material must be such that the secondary packaging can be given the necessary shape and the secondary packaging can perform the function required of it.

It is conventional in the industry to use as materials plastics selected from among the polyolefins (poly(ethylene), poly(propylene)), the polystyrenes, the polycarbonates, the polyamides, the polyurethanes and the polyesters. These have the necessary rigidity and mobility to perform the mechanical functions. Their disadvantage is that they are permeable to moisture from the air, as a result of their method of construction. There is therefore a need to increase the ability of the packaging to provide stable storage for the inhalable powder.

### DESCRIPTION OF THE INVENTION

The invention is therefore based on the problem of providing a powder inhaler having improved properties during long-term storage and during in-use storage of moisture-sensitive medicament formulations.

The powder inhaler according to the invention is characterised in that a dewatering material is incorporated at least in part of the powder inhaler.

The invention also relates to the use of the powder inhaler according to the invention for administering moisture-sensitive inhalable medicament formulations.

### DETAILED DESCRIPTION OF THE INVENTION

Powder inhalers are known from the prior art, as described hereinbefore. With the powder inhaler according to the invention, a moisture-sensitive inhalable formulation which has to be stored for lengthy periods in a powder inhaler before being administered is better protected from the penetration of moisture from the outer environment than is the case with comparable powder inhalers known from the prior art.

The powder inhaler according to the invention consists of dewatering material, at least in one or more parts. Parts of the powder inhaler may be for example the outer wall, the capsule holder, the capsule chamber or the blister disc. Preferably, the dewatering material is incorporated in a wall of the housing of the powder inhaler, most preferably in the capsule chamber (as for example in the Handihaler device) or in the wall of the reservoir of a reservoir device.

The present invention preferably relates to an assembly comprising an inhaler for inhaling powdered medicaments with a dewatering material, wherein the inhaler is characterised by a) un upwardly open, cup-shaped lower part (1) which has two opposing ports (2) in its casing and at the edge of the opening has a first hinge element with a joint pin (3), b) a plate (9) that covers the opening of the lower part (1) and comprises a second hinge element, as well as a screen holder (11) with a screen (10), c) a countersinkable capsule holder (4) for accommodating the capsule, which is constructed perpendicularly to the plane of the plate on the side of the plate (9) facing the lower part, and on which is provided a head that is movable counter to a spring, the head being provided with one or two sharpened pins (6), d) a mouthpiece (12) with a mouth tube and optionally a gripping aid (17) and a third hinge element, as well as e) a cover (13) that comprises a fourth hinge element, the hinge elements (one) of the lower part, (two) of the plate, (three) of the upper part and (four) of the cover being joined together. In addition, the inhaler comprises an actuating member (7) which serves to open the cover (13) as a result of the closure element (14) on the cover (13) striking the sloping side wall (15) (optionally provided with grooves (16)) of the recess (8), which acts as a sliding surface and releases the cover (13) as the actuating member (7) continues to advance.

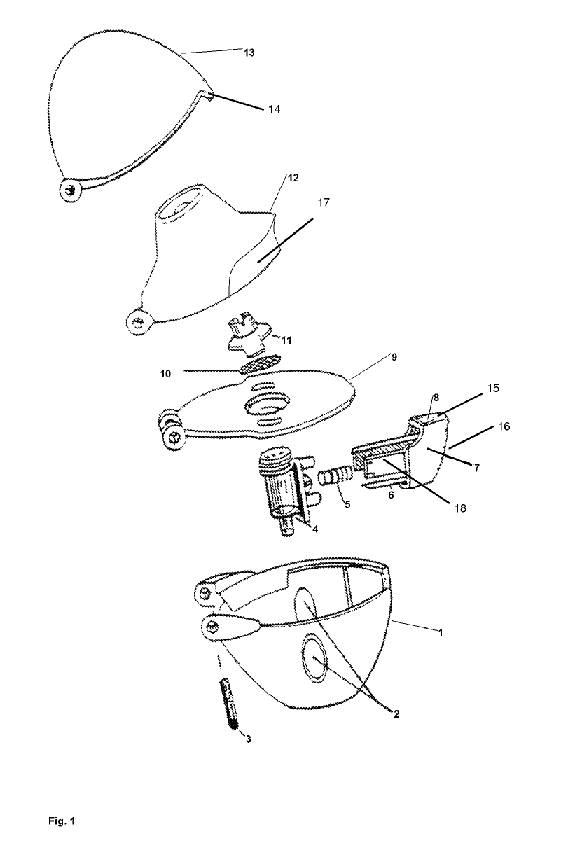

The guiding of the pin or pins is substantially carried out by means of two laterally mounted guide arms (18). The guide arms also have the task of holding the actuating member (7) under prestressing. For this purpose the guide arms (18) are provided with end stops at their end remote from the main body, which abut on the guide sleeves of the capsule holder (4) in the resting position of the actuating member (7). The guide sleeves are arranged on the outside of the capsule holder (4). Between the guide arms (18) is mounted a helical spring (5) which extends in its axial direction parallel to the pin or pins (6), the helical spring (5) being matched to the length of the guide arms (18) in such a way that the actuating member (7) is also biased into the resting position. An inhaler of this kind is shown in FIG. 1.

The present invention also preferably relates to an active multi-dose powder inhaler as disclosed in PCT/EP2007/004417, having a dewatering material. In this inhaler, the cover of the mouthpiece is to be coupled to a conveying device such as a pump and/or to an energy store such as a spring-type storage means such that by opening and/or closing the cover the conveying device is actuated and/or energy is generated and stored in the energy store. Particularly when the cover is opened, a conveying medium, preferably air, is taken in and/or put under pressure by the conveying means. Alternatively or additionally the energy generated by opening and/or closing the cover is preferably stored by biasing the spring-type storage means. This provides very simple, particularly intuitive operation of the inhaler. Similarly, it results in a particularly simple and hence inexpensive construction. For example it does away with the need for a separate actuating element for operating the conveying device or pump and/or for biasing the spring-type storage means or the like.

The inhaler may also have a gear for converting the opening and/or closing movement of the cover into a preferably axial movement for opening the next receptacle, for moving and/or advancing the store by one receptacle, for biasing a spring-type storage means, for actuating a conveying device, particularly taking in air, and/or for operating a counter or other device within the inhaler. The conveying device of the energy store and/or an ancillary device may be disposed within an annular store or an annular arrangement of receptacles each of which contains a dose of the formulation.

The present invention also preferably relates to a passive multi-dose powder inhaler as disclosed in PCT/EP2007/004416, with a dewatering material. In this inhaler, the carrier extends over a circumferential angle of the inhaler of less than 360°, is guided between two deflectors each with an at least substantially constant curvature, extends solely in an annular segment of the inhaler and/or extends with one of two sections connecting the deflectors exclusively along a circumferential or outer wall of the inhaler. The carrier is in the shape of a ribbon and/or a blister strip. The receptacles are preferably formed by blister pouches. The inhaler also comprises a conveying device with a plurality of wheels for advancing and/or deflecting the carrier stepwise. The wheels have the same diameter, are arranged on a common radius, can be driven by common drive means, particularly a sun wheel or the like, and/or have the same direction of rotation.

Furthermore the dewatering material can be incorporated in the blister disc. Specifically excluded in this context are capsules used as primary packaging and blisters filled with an inhalable formulation for use in powder inhalers: from DE 10 2005 022 862.3 capsules are known as primary packaging which contain an adsorbent in their walls. From EP 04 025 038.3 blisters for use in inhalers are known which have a dewatering agent in their walls.

Inhalable formulations here are preferably pharmaceutical powder formulations which contain an anticholinergic as active ingredient and the particles of which are less than 100 microns in size.

The compounds listed below may be used in the device according to the invention on their own or in combination. In the compounds mentioned below, W is a pharmacologically active substance and is selected (for example) from among the betamimetics, anticholinergics, corticosteroids, PDE4-inhibitors, LTD4-antagonists, EGFR-inhibitors, dopamine agonists, H1-antihistamines, PAF-antagonists and PI3-kinase inhibitors. Moreover, double or triple combinations of W may be formulated and used in the device according to the invention. Combinations of W might be, for example:
W denotes a betamimetic, combined with an anticholinergic, corticosteroid, PDE4-inhibitor, EGFR-inhibitor or LTD4-antagonist,
W denotes an anticholinergic, combined with a betamimetic, corticosteroid, PDE4-inhibitor, EGFR-inhibitor or LTD4-antagonist,
W denotes a corticosteroid, combined with a PDE4-inhibitor, EGFR-inhibitor or LTD4-antagonist
W denotes a PDE4-inhibitor, combined with an EGFR-inhibitor or LTD4-antagonist
W denotes an EGFR-inhibitor, combined with an LTD4-antagonist.

The compounds used as betamimetics are preferably compounds selected from among albuterol, arformoterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sulphonterol, terbutaline, tiaramide, tolubuterol, zinterol, CHF-1035, HOKU-81, KUL-1248 and
3-(4-{6-[2-hydroxy-2-(4-hydroxy-3-hydroxymethyl-phenyl)-ethylamino]-hexyloxy}-butyl)-benzyl-sulphonamide
5-[2-(5,6-diethyl-indan-2-ylamino)-1-hydroxy-ethyl]-8-hydroxy-1H-quinolin-2-one
4-hydroxy-7-[2-{[2-{[3-(2-phenylethoxy)propyl]sulphonyl}ethyl]-amino}ethyl]-2(3H)-benzothiazolone
1-(2-fluoro-4-hydroxyphenyl)-2-[4-(1-benzimidazolyl)-2-methyl-2-butylamino]ethanol
1-[3-(4-methoxybenzyl-amino)-4-hydroxyphenyl]-2-[4-(1-benzimidazolyl)-2-methyl-2-butylamino]ethanol
1-[2H-5-hydroxy-3-oxo-4H-1,4-benzoxazin-8-yl]-2-[3-(4-N,N-dimethylaminophenyl)-2-methyl-2-propylamino]ethanol
1-[2H-5-hydroxy-3-oxo-4H-1,4-benzoxazin-8-yl]-2-[3-(4-methoxyphenyl)-2-methyl-2-propylamino]ethanol
1-[2H-5-hydroxy-3-oxo-4H-1,4-benzoxazin-8-yl]-2-[3-(4-n-butyloxyphenyl)-2-methyl-2-propylamino]ethanol
1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol
5-hydroxy-8-(1-hydroxy-2-isopropylaminobutyl)-2H-1,4-benzoxazin-3-(4H)-one
1-(4-amino-3-chloro-5-trifluoromethylphenyl)-2-tert.-butylamino)ethanol
6-hydroxy-8-{1-hydroxy-2-[2-(4-methoxy-phenyl)-1,1-dimethyl-ethylamino]-ethyl}-4H-benzo[1,4]oxazin-3-one
6-hydroxy-8-{1-hydroxy-2-[2-(ethyl 4-phenoxy-acetate)-1,1-dimethyl-ethylamino]-ethyl}-4H-benzo[1,4]oxazin-3-one
6-hydroxy-8-{1-hydroxy-2-[2-(4-phenoxy-acetic acid)-1,1-dimethyl-ethylamino]-ethyl}-4H-benzo[1,4]oxazin-3-one
8-{2-[1,1-dimethyl-2-(2,4,6-trimethylphenyl)-ethylamino]-1-hydroxy-ethyl}-6-hydroxy-4H-benzo[1,4]oxazin-3-one
6-hydroxy-8-{1-hydroxy-2-[2-(4-hydroxy-phenyl)-1,1-dimethyl-ethylamino]-ethyl}-4H-benzo[1,4]oxazin-3-one
6-hydroxy-8-{1-hydroxy-2-[2-(4-isopropyl-phenyl)-1,1-dimethyl-ethylamino]-ethyl}-4H-benzo[1,4]oxazin-3-one
8-{2-[2-(4-ethyl-phenyl)-1,1-dimethyl-ethylamino]-1-hydroxy-ethyl}-6-hydroxy-4H-benzo[1,4]oxazin-3-one
8-{2-[2-(4-ethoxy-phenyl)-1,1-dimethyl-ethylamino]-1-hydroxy-ethyl}-6-hydroxy-4H-benzo[1,4]oxazin-3-one
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid
8-{2-[2-(3,4-difluoro-phenyl)-1,1-dimethyl-ethylamino]-1-hydroxy-ethyl}-6-hydroxy-4H-benzo[1,4]oxazin-3-one
1-(4-ethoxy-carbonylamino-3-cyano-5-fluorophenyl)-2-(tert-butylamino)ethanol
2-hydroxy-5-(1-hydroxy-2-{2-[4-(2-hydroxy-2-phenyl-ethylamino)-phenyl]-ethylamino}-ethyl)-benzaldehyde
N-[2-hydroxy-5-(1-hydroxy-2-{2-[4-(2-hydroxy-2-phenyl-ethylamino)-phenyl]-ethylamino}-ethyl)-phenyl]-formamide
8-hydroxy-5-(1-hydroxy-2-{2-[4-(6-methoxy-biphenyl-3-ylamino)-phenyl]-ethylamino}-ethyl)-1H-quinolin-2-one
8-hydroxy-5-[1-hydroxy-2-(6-phenethylamino-hexylamino)-ethyl]-1H-quinolin-2-one
5-[2-(2-{4-[4-(2-amino-2-methyl-propoxy)-phenylamino]-phenyl}-ethylamino)-1-hydroxy-ethyl]-8-hydroxy-1H-quinolin-2-one
[3-(4-{6-[2-hydroxy-2-(4-hydroxy-3-hydroxymethyl-phenyl)-ethylamino]-hexyloxy}-butyl)-5-methyl-phenyl]-urea
4-(2-{6-[2-(2,6-dichloro-benzyloxy)-ethoxy]-hexylamino}-1-hydroxy-ethyl)-2-hydroxymethyl-phenol
3-(4-{6-[2-hydroxy-2-(4-hydroxy-3-hydroxymethyl-phenyl)-ethylamino]-hexyloxy}-butyl)-benzylsulphonamide
3-(3-{7-[2-hydroxy-2-(4-hydroxy-3-hydroxymethyl-phenyl)-ethylamino]-heptyloxy}-propyl)-benzylsulphonamide
4-(2-{6-[4-(3-cyclopentanesulphonyl-phenyl)-butoxy]-hexylamino}-1-hydroxy-ethyl)-2-hydroxymethyl-phenol
N-Adamantan-2-yl-2-(3-{2-[2-hydroxy-2-(4-hydroxy-3-hydroxymethyl-phenyl)-ethylamino]-propyl}-phenyl)-acetamide
   optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The anticholinergics used are preferably compounds selected from among the tiotropium salts, preferably the bromide salt, oxitropium salts, preferably the bromide salt, flutropium salts, preferably the bromide salt, ipratropium salts, preferably the bromide salt, glycopyrronium salts, preferably the bromide salt, trospium salts, preferably the chloride salt, tolterodine. In the above-mentioned salts the cations are the pharmacologically active constituents. As anions the above-mentioned salts may preferably contain the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred.

Other preferred anticholinergics are selected from among the salts of formula AC-1

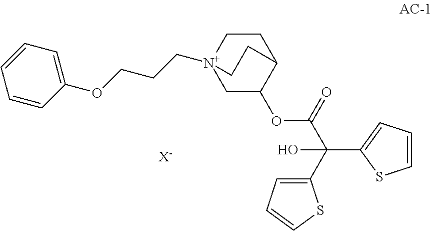

wherein X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate, preferably an anion with a single negative charge, particularly preferably an anion selected from among the fluoride, chloride, bromide, methanesulphonate and p-toluenesulphonate, particularly preferably bromide, optionally in the form of the racemates, enantiomers or hydrates thereof. Of particular importance are those pharmaceutical combinations which contain the enantiomers of formula AC-1-en

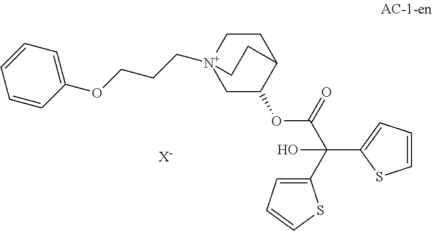

wherein X⁻ may have the above-mentioned meanings. Other preferred anticholinergics are selected from the salts of formula AC-2

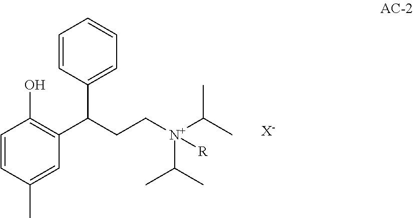

wherein R denotes either methyl or ethyl and wherein X⁻ may have the above-mentioned meanings. In an alternative embodiment the compound of formula AC-2 may also be present in the form of the free base AC-2-base.

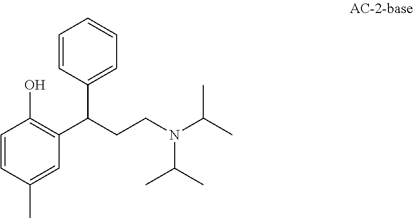

Other specified compounds are:
tropenol 2,2-diphenylpropionate methobromide,
scopine 2,2-diphenylpropionate methobromide,
scopine 2-fluoro-2,2-diphenylacetate methobromide,
tropenol 2-fluoro-2,2-diphenylacetate methobromide;
tropenol 3,3′,4,4′-tetrafluorobenzilate methobromide,
scopine 3,3′,4,4′-tetrafluorobenzilate methobromide,
tropenol 4,4′-difluorobenzilate methobromide,
scopine 4,4′-difluorobenzilate methobromide,
tropenol 3,3′-difluorobenzilate methobromide,
scopine 3,3′-difluorobenzilate methobromide;
tropenol 9-hydroxy-fluorene-9-carboxylate methobromide;
tropenol 9-fluoro-fluorene-9-carboxylate methobromide;
scopine 9-hydroxy-fluorene-9-carboxylate methobromide;
scopine 9-fluoro-fluorene-9-carboxylate methobromide;
tropenol 9-methyl-fluorene-9-carboxylate methobromide;
scopine 9-methyl-fluorene-9-carboxylate methobromide;
cyclopropyltropine benzilate methobromide;
cyclopropyltropine 2,2-diphenylpropionate methobromide;
cyclopropyltropine 9-hydroxy-xanthene-9-carboxylate methobromide;
cyclopropyltropine 9-methyl-fluorene-9-carboxylate methobromide;
cyclopropyltropine 9-methyl-xanthene-9-carboxylate methobromide;
cyclopropyltropine 9-hydroxy-fluorene-9-carboxylate methobromide;
cyclopropyltropine methyl 4,4′-difluorobenzilate methobromide.
tropenol 9-hydroxy-xanthene-9-carboxylate methobromide;
scopine 9-hydroxy-xanthene-9-carboxylate methobromide;
tropenol 9-methyl-xanthene-9-carboxylate-methobromide;
scopine 9-methyl-xanthene-9-carboxylate-methobromide;
tropenol 9-ethyl-xanthene-9-carboxylate methobromide;
tropenol 9-difluoromethyl-xanthene-9-carboxylate methobromide;
scopine 9-hydroxymethyl-xanthene-9-carboxylate methobromide,

The above-mentioned compounds may also be used as salts within the scope of the present invention, wherein instead of the methobromide the salts metho-X are used, wherein X may have the meanings given hereinbefore for X.

As corticosteroids it is preferable to use compounds selected from among beclomethasone, betamethasone, budesonide, butixocort, ciclesonide, deflazacort, dexamethasone, etiprednol, flunisolide, fluticasone, loteprednol, mometasone, prednisolone, prednisone, rofleponide, triamcinolone, RPR-106541, NS-126, ST-26 and
(S)-fluoromethyl 6,9-difluoro-17-[(2-furanylcarbonyl)oxy]-11-hydroxy-16-methyl-3-oxo-androsta-1,4-diene-17-carbothionate
(S)-(2-oxo-tetrahydro-furan-3S-yl)6,9-difluoro-11-hydroxy-16-methyl-3-oxo-17-propionyloxy-androsta-1,4-diene-17-carbothionate,
cyanomethyl 6α,9α-difluoro-11β-hydroxy-16α-methyl-3-oxo-17α-(2,2,3,3-tertamethylcyclopropylcarbonyl)oxy-androsta-1,4-diene-17β-carboxylate
   optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, dichloroacetates, propionates, dihydrogen phosphates, palmitates, pivalates or furoates.

PDE4-inhibitors which may be used are preferably compounds selected from among enprofyllin, theophyllin, roflumilast, ariflo (cilomilast), tofimilast, pumafentrin, lirimilast, arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-11294A, C1-1018, CDC-801, CDC-3052, D-22888, YM-58997, Z-15370 and
N-(3,5-dichloro-1-oxo-pyridin-4-yl)-4-difluoromethoxy-3-cyclopropylmethoxybenzamide
(−)p-[4aR*,10bS*)-9-ethoxy-1,2,3,4,4a,10b-hexahydro-8-methoxy-2-methylbenzo[s][1,6]naphthyridin-6-yl]-N,N-diisopropylbenzamide
(R)-(+)-1-(4-bromobenzyl)-4-[(3-cyclopentyloxy)-4-methoxyphenyl]-2-pyrrolidone
3-(cyclopentyloxy-4-methoxyphenyl)-1-(4-N′-[N′-cyano-5-methyl-isothioureido]benzyl)-2-pyrrolidone
cis[4-cyano-4-(3-cyclopentyloxy-4-methoxyphenyl)cyclohexane-1-carboxylic acid]
2-carbomethoxy-4-cyano-4-(3-cyclopropylmethoxy-4-difluoromethoxy-phenyl)cyclohexan-1-one
cis[4-cyano-4-(3-cyclopropylmethoxy-4-difluoromethoxyphenyl)cyclohexan-1-ol]
(R)-(+)-ethyl[4-(3-cyclopentyloxy-4-methoxyphenyl)pyrrolidin-2-ylidene]acetate
(S)-(−)-ethyl[4-(3-cyclopentyloxy-4-methoxyphenyl)pyrrolidin-2-ylidene]acetate
9-cyclopentyl-5,6-dihydro-7-ethyl-3-(2-thienyl)-9H-pyrazolo[3,4-c]-1,2,4-triazolo[4,3-a]pyridine
9-cyclopentyl-5,6-dihydro-7-ethyl-3-(tert-butyl)-9H-pyrazolo[3,4-c]-1,2,4-triazolo[4,3-a]pyridine
   optionally in the form of the racemates, enantiomers or diastereomers thereof and optionally in the form of the pharmacologically acceptable acid addition salts thereof, the solvates and/or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The LTD4-antagonists used are preferably compounds selected from among montelukast, pranlukast, zafirlukast, MCC-847 (ZD-3523), MN-001, MEN-91507 (LM-1507), VUF-5078, VUF-K-8707, L-733321 and
1-(((R)-(3-(2-(6,7-difluoro-2-quinolinyl)ethenyl)phenyl)-3-(2-(2-hydroxy-2-propyl)phenyl)thio)methylcyclopropane-acetic acid,
1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid
[2-[[2-(4-tert-butyl-2-thiazolyl)-5-benzofuranyl]oxymethyl]phenyl]acetic acid
   optionally in the form of the racemates, enantiomers or diastereomers thereof and optionally in the form of the pharmacologically acceptable acid addition salts, solvates and/or hydrates thereof. According to the invention the acid addition salts of the betamimetics are preferably selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which the LTD4-antagonists may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

EGFR-inhibitors which may be used are preferably compounds selected from among cetuximab, trastuzumab, ABX-EGF, Mab ICR-62 and
4-[(3-chloro-4-fluorophenyl)amino]-6-{[4-(morpholin-4-yl)-1-oxo-2-buten-1-yl]-amino}-7-cyclopropylmethoxy-quinazoline
4-[(3-chloro-4-fluorophenyl)amino]-6-{[4-(N,N-diethylamino)-1-oxo-2-buten-1-yl]-amino}-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopropylmethoxy-quinazoline
4-[(R)-(1-phenyl-ethyl)amino]-6-{[4-(morpholin-4-yl)-1-oxo-2-buten-1-yl]amino}-7-cyclopentyloxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline
4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline
4-[(3-chloro-4-fluoro-phenyl)amino]-6-[2-((S)-6-methyl-2-oxo-morpholin-4-yl)-ethoxy]-7-methoxy-quinazoline
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopentyloxy-quinazoline
4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline
4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline
4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline
4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline
4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline
4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline
4-[(3-chloro-4-fluorophenyl)amino]-6-{[4-(N-cyclopropyl-N-methyl-amino)-1-oxo-2-buten-1-yl]amino}-7-cyclopentyloxy-quinazoline
4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline
4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline
4-[(3-ethynyl-phenyl)amino]-6,7-bis-(2-methoxy-ethoxy)-quinazoline
4-[(3-chloro-4-fluorophenyl)amino]-7-[3-(morpholin-4-yl)-propyloxy]-6-[(vinyl-carbonyl)amino]-quinazoline
4-[(R)-(1-phenyl-ethyl)amino]-6-(4-hydroxy-phenyl)-7H-pyrrolo[2,3-d]pyrimidine
3-cyano-4-[(3-chloro-4-fluorophenyl)amino]-6-{[4-(N,N-dimethylamino)-1-oxo-2-buten-1-yl]amino}-7-ethoxy-quinoline
4-{[3-chloro-4-(3-fluoro-benzyloxy)-phenyl]amino}-6-(5-{[(2-methanesulphonyl-ethyl)amino]methyl}-furan-2-yl)quinazoline
4-[(R)-(1-phenyl-ethyl)amino]-6-{[4-((R)-6-methyl-2-oxo-morpholin-4-yl)-1-oxo-2-buten-1-yl]amino}-7-methoxy-quinazoline
4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline
4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline
4-[(3-ethynyl-phenyl)amino]-6-{[4-(5,5-dimethyl-2-oxo-morpholin-4-yl)-1-oxo-2-buten-1-yl]amino}-quinazoline
4-[(3-chloro-4-fluoro-phenyl)amino]-6-[2-(2,2-dimethyl-6-oxo-morpholin-4-yl)-ethoxy]-7-methoxy-quinazoline
4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline
4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline
4-[(3-chloro-4-fluoro-phenyl)amino]-6-{2-[4-(2-oxo-morpholin-4-yl)-piperidin-1-yl]-ethoxy}-7-methoxy-quinazoline
4-[(3-chloro-4-fluoro-phenyl)amino]-6-[1-(tert.-butyloxycarbonyl)-piperidin-4-yloxy]-7-methoxy-quinazoline
4-[(3-chloro-4-fluoro-phenyl)amino]-6-(trans-4-amino-cyclohexan-1-yloxy)-7-methoxy-quinazoline
4-[(3-chloro-4-fluoro-phenyl)amino]-6-(trans-4-methanesulphonylamino-cyclohexan-1-yloxy)-7-methoxy-quinazoline
4-[(3-chloro-4-fluoro-phenyl)amino]-6-(tetrahydropyran-3-yloxy)-7-methoxy-quinazoline
4-[(3-chloro-4-fluoro-phenyl)amino]-6-(1-methyl-piperidin-4-yloxy)-7-methoxy-quinazoline
4-[(3-chloro-4-fluoro-phenyl)amino]-6-{1-[(morpholin-4-yl)carbonyl]-piperidin-4-yl-oxy}-7-methoxy-quinazoline
4-[(3-chloro-4-fluoro-phenyl)amino]-6-{1-[(methoxymethyl)carbonyl]-piperidin-4-yl-oxy}-7-methoxy-quinazoline
4-[(3-chloro-4-fluoro-phenyl)amino]-6-(piperidin-3-yloxy)-7-methoxy-quinazoline
4-[(3-chloro-4-fluoro-phenyl)amino]-6-[1-(2-acetylamino-ethyl)-piperidin-4-yloxy]-7-methoxy-quinazoline
4-[(3-chloro-4-fluoro-phenyl)amino]-6-(tetrahydropyran-4-yloxy)-7-ethoxy-quinazoline
4-[(3-chloro-4-fluoro-phenyl)amino]-6-((S)-tetrahydrofuran-3-yloxy)-7-hydroxy-quinazoline
4-[(3-chloro-4-fluoro-phenyl)amino]-6-(tetrahydropyran-4-yloxy)-7-(2-methoxy-ethoxy)-quinazoline
4-[(3-chloro-4-fluoro-phenyl)amino]-6-{trans-4-[(dimethylamino)sulphonylamino]-cyclohexan-1-yloxy}-7-methoxy-quinazoline
4-[(3-chloro-4-fluoro-phenyl)amino]-6-{trans-4-[(morpholin-4-yl)carbonylamino]-cyclohexan-1-yloxy}-7-methoxy-quinazoline
4-[(3-chloro-4-fluoro-phenyl)amino]-6-{trans-4-[(morpholin-4-yl)sulphonylamino]-cyclohexan-1-yloxy}-7-methoxy-quinazoline
4-[(3-chloro-4-fluoro-phenyl)amino]-6-(tetrahydropyran-4-yloxy)-7-(2-acetylamino-ethoxy)-quinazoline
4-[(3-chloro-4-fluoro-phenyl)amino]-6-(tetrahydropyran-4-yloxy)-7-(2-methanesulphonylamino-ethoxy)-quinazoline
4-[(3-chloro-4-fluoro-phenyl)amino]-6-{1-[(piperidin-1-yl)carbonyl]-piperidin-4-yloxy}-7-methoxy-quinazoline
4-[(3-chloro-4-fluoro-phenyl)amino]-6-(1-aminocarbonylmethyl-piperidin-4-yloxy)-7-methoxy-quinazoline
4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline
4-[(3-chloro-4-fluoro-phenyl)amino]-6-(trans-4-ethansulphonylamino-cyclohexan-1-yloxy)-7-methoxy-quinazoline
4-[(3-chloro-4-fluoro-phenyl)amino]-6-(1-methanesulphonyl-piperidin-4-yloxy)-7-ethoxy-quinazoline
4-[(3-chloro-4-fluoro-phenyl)amino]-6-(1-methanesulphonyl-piperidin-4-yloxy)-7-(2-methoxy-ethoxy)-quinazoline
4-[(3-chloro-4-fluoro-phenyl)amino]-6-[1-(2-methoxy-acetyl)-piperidin-4-yloxy]-7-(2-methoxy-ethoxy)-quinazoline
4-[(3-chloro-4-fluoro-phenyl)amino]-6-(cis-4-acetylamino-cyclohexan-1-yloxy)-7-methoxy-quinazoline
4-[(3-ethynyl-phenyl)amino]-6-[1-(tert.-butyloxycarbonyl)-piperidin-4-yloxy]-7-methoxy-quinazoline
4-[(3-ethynyl-phenyl)amino]-6-(tetrahydropyran-4-yloxy]-7-methoxy-quinazoline
4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline
4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline
4-[(3-chloro-4-fluoro-phenyl)amino]-6-{cis-4-[(morpholin-4-yl)carbonylamino]-cyclohexan-1-yloxy}-7-methoxy-quinazoline
4-[(3-chloro-4-fluoro-phenyl)amino]-6-{1-[2-(2-oxopyrrolidin-1-yl)ethyl]-piperidin-4-yloxy}-7-methoxy-quinazoline
4-[(3-chloro-4-fluoro-phenyl)amino]-6-{1-[(morpholin-4-yl)carbonyl]-piperidin-4-yloxy}-7-(2-methoxy-ethoxy)-quinazoline
4-[(3-ethynyl-phenyl)amino]-6-(1-acetyl-piperidin-4-yloxy)-7-methoxy-quinazoline
4-[(3-ethynyl-phenyl)amino]-6-(1-methyl-piperidin-4-yloxy)-7-methoxy-quinazoline
4-[(3-ethynyl-phenyl)amino]-6-(1-methanesulphonyl-piperidin-4-yloxy)-7-methoxy-quinazoline
4-[(3-chloro-4-fluoro-phenyl)amino]-6-(1-methyl-piperidin-4-yloxy)-7(2-methoxy-ethoxy)-quinazoline
4-[(3-chloro-4-fluoro-phenyl)amino]-6-(1-isopropyloxycarbonyl-piperidin-4-yloxy)-7-methoxy-quinazoline
4-[(3-chloro-4-fluoro-phenyl)amino]-6-(cis-4-methylamino-cyclohexan-1-yloxy)-7-methoxy-quinazoline
4-[(3-chloro-4-fluoro-phenyl)amino]-6-{cis-4-[N-(2-methoxy-acetyl)-N-methyl-amino]-cyclohexan-1-yloxy}-7-methoxy-quinazoline
4-[(3-ethynyl-phenyl)amino]-6-(piperidin-4-yloxy)-7-methoxy-quinazoline
4-[(3-ethynyl-phenyl)amino]-6-[1-(2-methoxy-acetyl)-piperidin-4-yloxy]-7-methoxy-quinazoline
4-[(3-ethynyl-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[cis-2,6-dimethyl-morpholin-4-yl)carbonyl]-piperidin-4-yloxy}-7-methoxy-quinazoline
4-[(3-chloro-4-fluoro-phenyl)amino]-6-{1-[(2-methyl-morpholin-4-yl)carbonyl]-piperidin-4-yloxy}-7-methoxy-quinazoline
4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline
4-[(3-chloro-4-fluoro-phenyl)amino]-6-{1-[(N-methyl-N-2-methoxyethyl-amino)carbonyl]-piperidin-4-yloxy}-7-methoxy-quinazoline
4-[(3-chloro-4-fluoro-phenyl)amino]-6-(1-ethyl-piperidin-4-yloxy)-7-methoxy-quinazoline
4-[(3-chloro-4-fluoro-phenyl)amino]-6-{1-[(2-methoxyethyl)carbonyl]-piperidin-4-yloxy}-7-methoxy-quinazoline
4-[(3-chloro-4-fluoro-phenyl)amino]-6-{1-[(3-methoxypropyl-amino)-carbonyl]-piperidin-4-yloxy}-7-methoxy-quinazoline
4-[(3-chloro-4-fluoro-phenyl)amino]-6-[cis-4-(N-methanesulphonyl-N-methyl-amino)-cyclohexan-1-yloxy]-7-methoxy-quinazoline
4-[(3-chloro-4-fluoro-phenyl)amino]-6-[cis-4-(N-acetyl-N-methyl-amino)-cyclohexan-1-yloxy]-7-methoxy-quinazoline
4-[(3-chloro-4-fluoro-phenyl)amino-]-6-(trans-4-methylamino-cyclohexan-1-yloxy)-7-methoxy-quinazoline
4-[(3-chloro-4-fluoro-phenyl)amino-]-6-{trans-4-(N-methanesulphonyl-N-methyl-amino)-cyclohexan-1-yloxy}-7-methoxy-quinazoline
4-[(3-chloro-4-fluoro-phenyl)amino-]-6-(trans-4-dimethylamino-cyclohexan-1-yloxy)-7-methoxy-quinazoline
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline
4-[(3-chloro-4-fluoro-phenyl)amino-]-6-[2-(2,2-dimethyl-6-oxo-morpholin-4-yl)-ethoxy]-7-[(S)-(tetrahydrofuran-2-yl)methoxy]-quinazoline
4-[(3-chloro-4-fluoro-phenyl)amino]-6-(1-methanesulphonyl-piperidin-4-yloxy)-7-methoxy-quinazoline
4-[(3-chloro-4-fluoro-phenyl)amino]-6-(1-cyano-piperidin-4-yloxy)-7-methoxy-quinazoline
   optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The dopamine agonists used are preferably compounds selected from among bromocriptin, cabergoline, alpha-dihydroergocryptine, lisuride, pergolide, pramipexol, roxindol, ropinirol, talipexol, tergurid and viozan, optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate.

H1-Antihistamines which may be used are preferably compounds selected from among epinastine, cetirizine, azelastine, fexofenadine, levocabastine, loratadine, mizolastine, ketotifen, emedastine, dimetindene, clemastine, bamipine, cexchlorpheniramine, pheniramine, doxylamine, chlorophenoxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

It is also possible to use inhalable macromolecules, as disclosed in EP 1 003 478.

In addition, the compound may come from the groups of ergot alkaloid derivatives, the triptans, the CGRP-inhibitors, the phosphodiesterase-V inhibitors, optionally in the form of the racemates, enantiomers or diastereomers thereof, optionally in the form of the pharmacologically acceptable acid addition salts, the solvates and/or hydrates thereof.

Examples of ergot alkaloid derivatives are dihydroergotamine and ergotamine.

Preferably, the material for the powder inhaler according to the invention may be polymer compositions which contain at least one thermoplastic polymer, at least one dewatering agent and optionally at least one elastomer and/or plasticiser and/or other fibres. The material contains no gelatine or cellulose or starch or derivatives thereof.

Preferred polymer compositions consist for example of
60-80 wt. % of one or more thermoplastic polymers,
b) 20-40 wt. % of one or more dewatering agents,
other substances

Preferably the amount of dewatering agent is 10-40 wt. %, more preferably 20-30 wt. %.

The polymer component of the plastics may be in particular thermoplastic polymers such as e.g. polystyrenes, polyolefins, polyamides, polyvinyl chlorides or polyurethanes. Particularly preferred are polyethylene (Hostalen), particularly polyethylene with a density of between 900 and 1000 kg/m³, preferably from 940-980 kg/m³, particularly preferably 960 kg/m³ (high-density polyethylene), polycarbonate, polyester, polypropylene or polyethylene terephthalate.

Dewatering agents that may be used include for example silica gels, zeolites, aluminium oxide, magnesium sulphate, molecular sieves etc.

Finally the polymer composition may also contain other inorganic or organic additives that have the following functions: plasticisers, stabilisers, dyes, pigments or the like.

Preferably dewatering materials—i.e. Plastics that contain a dewatering agent—are used that can be processed by injection moulding or blow moulding. Also preferred are plastics whose processing does not require the use of a mould release agent which may cause the filling, i.e. the pharmaceutical formulation, to adhere to the walls. This has the advantage that the interior of the receptacle does not have to be cleaned to remove mould release agent in order to satisfy the statutory requirements (e.g. According to the DAB (German Pharmacopoeia)) which restrict the use of mould release agents for primary packaging.

In a preferred embodiment the dewatering material does not exhibit any marked adhesion for pharmaceutical/chemical substances, particularly for particles of a size intended to enter the lungs. This ensures more accurate dosing, particularly of the lung-bound fine fraction of the pharmaceutical preparation.

Further information relating to the composition or processing may be found in the prior art, particularly EP 599690, EP 432438 or EP 400460.

In one embodiment the walls of the inhaler component may contain regions with a different composition of polymer/dewatering agent.

In other embodiments the walls of the inhaler component consist of at least two layers, an inner layer and at least one outer layer thereon. One layer of the inhaler component then consists of a polymer composition without any dewatering agent, while the other layer contains a dewatering agent.

The powder inhaler according to the invention offers advantages above all when active substances, adjuvants or formulations are to be protected from the uptake of water. For example this applies to inhalable powders which have been prepared by spray drying and/or for active substances, adjuvants and formulations which are in an amorphous state.

A particularly preferred inhaler according to the invention is for example a device known by the brand name HandiHaler®, as disclosed e.g. in EP 1342483. A preferred embodiment of this aspect of the invention relates to an assembly comprising an inhaler for the inhalation of powdered pharmaceutical compositions and a two-part capsule, wherein the inhaler is characterised by a) a) un upwardly open, cup-shaped lower part which has two opposing ports in its casing and at the edge of the opening has a first hinge element with a joint pin, b) a plate that covers the opening of the lower part and comprises a second hinge element, c) an inhalation chamber for accommodating the capsule, which is constructed perpendicularly to the plane of the plate on the side of the plate facing the lower part, and on which is provided a head that is movable counter to a spring, the head being provided with two sharpened pins, d) a mouthpiece with a mouth tube and a third hinge element, as well as e) a cover that comprises a fourth hinge element, the hinge elements (one) of the lower part, (two) of the plate, (three) of the upper part and (four) of the cover being joined together.

### EXAMPLES

The water permeation through the container walls of the reservoirs and the possible amount of water that could be retained by a dewatering agent incorporated therein was calculated by way of example for five multi-dose powder inhalers. Table 1 shows the mass, wall thickness and surface area for all the relevant inhaler components.

TABLE 1
Dimensions of the inhaler components

| | | | | Surface | Wall |
|---|---|---|---|---|---|
| | | | Mass | area | thickness |
| Inhaler | | Polymer | [g] | [cm²] | [cm] |
| Budefat | white component | poly(alkylterephthalate) | 1.5 | 7.73 | 0.113 |
| | round component | poly(propylene) atact. | 0.2 | 0.84 | 0.076 |
| | pointed component | poly(propylene) atact. | 0.5 | 3.77 | 0.111 |
| CertiHaler | container | poly(alkylterephthalate) | 1.5 | 9.27 | 0.115 |
| | cover | poly(alkylterephthalate) | 0.8 | 4.62 | 0.800 |
| EasyHaler | container | poly(carbonate) | 2.9 | 21.13 | 0.120 |
| | cover | poly(propylene) atact. | 0.8 | 1.77 | 0.090 |
| Novolizer | container | poly(styrene) | 2.1 | 18.78 | 0.137 |
| | cover | poly(propylene) atact. | 0.5 | 1.80 | 0.117 |
| Turbu- | opaque | poly(propylene) atact. | 1.4 | 8.69 | 0.074 |
| Haler | components | | | | |
| | perforated | poly(propylene) atact. | 0.5 | 2.79 | 0.062 |
| | component | | | | |

Based on two possible fill concentrations for the dewatering agent in the polymer (e.g. 10 and 40 wt. %), the water uptake capacities shown in Table 2 are obtained. The calculation is done using formula (1).

TABLE 2
Sum of the masses and water uptake capacities of housing components containing
the powder formulation in selected inhalers

| | Mass of | Mass of reservoir | water uptake | water uptake |
|---|---|---|---|---|
| | reservoir | container walls* | capacity (10 wt. | capacity (40 wt. |
| Inhaler | component [g] | [g] | %) [mg]** | %) [mg]** |
| Budefat | 2.7 | 2.2 | 44 | 175 |
| CertiHaler | 2.3 | 2.3 | 46 | 185 |
| EasyHaler | 6.7 | 3.7 | 74 | 295 |
| Novolizer | 4.0 | 2.6 | 52 | 210 |
| TurbuHaler | 1.9 | 1.8 | 36 | 145 |

*sum of several components in some cases
**water uptake capacity corresponds to 20% of the intrinsic weight of the dewatering agent

Calculation formula (1) for Table 2:

WK=m_{R}·x %·20% (1)

WK: Water uptake capacity [g]

m_{R}: Mass of the reservoir container wall

x: Percentage by weight of the dewatering agent

It is assumed that the permeation through the wall of the component is the only access route for water. Thus different amounts of water ingress (permeations) are obtained for the different polymers, and these are compared with the individual water uptake capacities in Table 3.

TABLE 3
Water uptake capacity of the inhaler components under consideration compared
with the permeation of water through the component wall

| | | | | water permeation* |
|---|---|---|---|---|
| | | | | through the |
| | | | | container wall |
| | | | water uptake | without dewatering |
| | | | capacity | agent |
| Inhaler | | polymer/mass [g] | (40 wt. %) [mg] | [mg] per month |
| Budefat | white | poly(alkylterephthalate) | 120 | 3.1 |
| | component | | | |
| | round | poly(propylene) atact. | 15 | 0.1 |
| | component | | | |
| | pointed | poly(propylene) atact. | 40 | 0.2 |
| | component | | | |
| CertiHaler | container | poly(alkylterephthalate) | 120 | 3.7 |
| | cover | poly(alkylterephthalate) | 65 | 2.7 |
| EasyHaler | container | poly(carbonate) | 230 | 8.1 |
| | cover | poly(propylene) atact. | 65 | 0.1 |
| Novolizer | container | poly(styrene) | 170 | 63.1 |
| | cover | poly(propylene) atact. | 40 | 0.1 |
| Turbu- | opaque | poly(propylene) atact. | 110 | 0.5 |
| Haler | component | | | |
| | perforated | poly(propylene) atact. | 35 | 0.2 |
| | component | | | |

*taking account of the wall thickness and the chemical identity of the polymer, permeation calculated according to Polymerhandbook (Brandrup, Wiley-Interscience, 1998) at 25° C. and 75% r.h. outside (0% r.h. Inside)

Calculation formula (2) for Table 3:

$$WP = \frac{Δ p \cdot A \cdot t \cdot P}{d} \cdot \frac{M ( {H}_{2} O )}{R \cdot T} \cdot {p}_{norm} \qquad ( 2 )$$

WP: water permeation [g]

Δp: Difference in steam pressure outside and inside the reservoir container, assumed here to be 2411 Pa, corresponding to 75% relative humidity at 25° C.)

A: Surface of the reservoir container component

t: Period of time for the permeation (assumed to be 30 days in this case)

d: Wall thickness of the reservoir container component

P: Permeation coefficient [cm²/(s Pa)]
P (poly(styrene))=10⁻¹⁰ cm²/(s·Pa)
P (poly(alkylterephthalate)/poly(carbonate))=10⁻¹¹ cm²/(s·Pa)
P (poly(propylene) atact.)=10⁻¹² cm²/(s·Pa)

M(H₂O): 18 g/mol, molar mass of water

R: 8.314 J/(K mol), gas constant

T: 298 K (25° C.), ambient temperature

Pₙₒᵣₘ: 101325 Pa, standard pressure

As can be seen from Table 3, the use of dewatering materials is highly relevant to protecting the formulation against moisture. Even minor amounts of the dewatering agent would protect the formulation in all inhalers, assuming an otherwise leaktight reservoir container. However, water that permeates through the actual openings provided in the reservoir containers can also be bound in large amounts by the use of dewatering materials in the container walls.

1. Powder inhaler that contains a moisture-sensitive inhalable formulation, characterised in that a dewatering material is incorporated at least in part of the powder inhaler.

2. Powder inhaler according to claim 1, characterised in that the dewatering material is incorporated in the walls of the housing.

3. Powder inhaler according to claim 1, characterised in that the dewatering material is incorporated in the capsule chamber of the powder inhaler.

4. Powder inhaler according to claim 1, characterised in that the dewatering material is incorporated in the blister disc of the powder inhaler.

5. Powder inhaler according to claim 1, characterised in that the dewatering material is incorporated in the walls of the reservoir container of a reservoir-type multi-dose powder inhaler.

6. Powder inhaler according to claim 1, characterised in that the powder inhaler consists of a polymer composition which contains at least one thermoplastic polymer, at least one dewatering agent, optionally at least one elastomer and/or optionally plasticisers and/or other fibres.

7. Powder inhaler according to claim 6, characterised in that the dewatering agent is present in an amount of from 10-40% wt. %.

8. Powder inhaler according to claim 7, characterised in that the dewatering agent is present in an amount of from 20-30% wt. %.

9. Powder inhaler according to claim 7, characterised in that the dewatering agent comprises silica gels, zeolites, aluminium oxide, magnesium sulphate, molecular sieves.

10. Powder inhaler according to claim 1, characterised in that it is a single-dose or multi-dose powder inhaler.

11. Powder inhaler according to claim 1, characterised in that the inhalable formulation is used for the treatment of respiratory complaints.

12. Powder inhaler according to claim 1, characterised in that the inhalable formulation is moisture-sensitive, containing an active substance selected from among anticholinergics, betamimetics, steroids, phosphodiesterase-IV-inhibitors, LTD4-antagonists, EGFR-kinase inhibitors, dopamine agonists, H1-antihistamines, PAF-antagonists, P13-kinase inhibitors, P38 MAP-kinase inhibitors, antiallergics, ergot alkaloid derivatives, triptans, CGRP-antagonists, phosphodiesterase-V-inhibitors, combinations of these active substances, and medicament formulations containing one or more of these active substances.
